(19) United States
(12) Patent Application Publication
(10) Pub. No.: US 2009/0024004 A1
(43) Pub. Date: Jan. 22, 2009

(54) Method and Apparatus for Monitoring Body Temperature, Respiration, Heart Sound, Swallowing, and Medical Inquiring

(76) Inventor: Chang-ming Yang, Miaoli (TW)

Correspondence Address: Dr. BANGER SHIA;Int''l Patent Office of Bang Shia, 102 Lindencrest Ct, Sugar Land, TX, 77479-5201, US

(21) Appl. No.: 11/718,095

(22) PCT Filed: Oct. 29, 2004
(86) PCT No.: PCT/CN2004/001235

§ 371 Date: Apr. 27, 2007

(51) Int. Cl. A61B 5/01; A61B 5/00

(52) U.S. Cl. 600/301

(57) ABSTRACT

The invention provides a method as well as a device for the monitoring of body temperature, respiration, heart sound, swallowing, and offering medical inquiries. It comprises a sensor, a monitoring device, and medical devices. Installing proper sensors on the user's body and making the users wear necessary medical devices. The sensors attached to the user's body will be sensing the physical conditions and send the data collected to the monitoring device for security and protection. It can also allow the user or the medical personnel conduct two-way inquiries or treatment through displaying device, or the monitoring device can control the medical devices and assist the treatment. Furthermore, the data can be transferred to the medical system in the distant end and the user's data and be updated immediately and reduce errors happened in keying in the data. Doctors will be able to proceed with medical treatment according to the data and prevent abrupt accidents in patients as well as further protecting the security of the user.

### BACKGROUND OF THE INVENTION

1. Field of the Invention

The present invention relates to a method and an apparatus for monitoring body temperature, respiration, heart sound, swallowing, and medical inquiring, and more particularly, to a method and an apparatus for monitoring a user's physical conditions, so as to perform suitable rescue solution when the user is in a critical condition.

2. Description of the Prior Art

The applicant of the invention has filed an application entitled “Apparatus for monitoring physical condition and the monitoring method thereof”, which provides an interactive model for monitoring user's instant physical condition. When the apparatus detected an abnormal condition of the user, it can warn the user or perform the role of an assistant in administering treatment. Particularly, when the user is in a critical condition, doctors can rescue the user immediately, and reduce the death rate caused by a waste of time.

The above-mentioned application has already provided good monitoring functions. However, the applicant makes further modification and studies and focuses particularly on the respiratory tract and lungs. This is because the respiratory tract diseases have become the main cause of sudden death in human beings (especially for children and elder people). Sudden death is mostly caused by acute conditions such as abnormal body temperature, disturbed respiration, or simply the obstruction of respiration tract caused by sputum. Moreover, a simple instruction of treatment may rescue a life from death caused by the above-mentioned accidents. Accordingly, the applicant realizes the importance of monitoring the body temperature, respiration, heartbeat, heart sound, swallowing, and further develops the apparatus for monitoring body temperature, respiration, heart sound, swallowing, and medical inquiring.

### SUMMARY OF THE INVENTION

Accordingly, an aspect of the present invention is to provide a method and an apparatus for monitoring body temperature, respiration, heart sound, swallowing, and medical inquiring. Particularly, the invention can monitor user's instant physical function through tiny sensors, and rescue the user immediately based on the results monitored, so as to reduce the death rate cause by a waste of time.

Another aspect of the invention is to provide a method and an apparatus for monitoring body temperature, respiration, heart sound, swallowing, and medical inquiring. Particularly, the aspect of the invention is to provide an interactive monitoring and rescuing model. The user who carries the apparatus of the invention can maintain their life quality, and can also get suitable medical assistance in time when they need it.

The apparatus of the invention includes a sensor, a monitoring device, and a medical device. The sensor is disposed on a monitoring position of an user, for delivering the information of physical condition of the user to the device. Furthermore, the sensor mainly monitors the user's respiratory tract, including body temperature and respiration. Because body temperature is related to a wide range of diseases, such as infection, it can therefore be applied to diagnose other diseases. The sensor further delivers the information to the monitoring device. The monitoring device receives the information of the physical condition of the user, and compares the information with a pre-stored data to determine the user's condition. Meanwhile, the monitoring device can also obtain reference information from different parts of the user's body, or further analyzes the medical history of the user to come up with more accurate determination. The medical device is disposed in a protective garment, a belt, an elastic belt of underpants, or an underwear worn by the user, for performing suitable rescuing measures to the user according to orders sent by the monitoring device.

The advantage and spirit of the invention may be understood by the following recitations together with the appended drawings.

### BRIEF DESCRIPTION OF THE APPENDED DRAWINGS

FIG. 1 shows a diagram of the apparatus of the invention;

FIG. 2 shows the first example of the disposition of the sensor of the invention;

FIG. 3 shows the second example of the disposition of the sensor of the invention;

FIG. 4 shows the third example of the disposition of the sensor of the invention;

FIG. 5 shows the fourth example of the disposition of the sensor of the invention;

FIG. 6 shows the fifth example of the disposition of the sensor of the invention;

FIG. 7 shows the sixth example of the disposition of the sensor of the invention;

FIG. 8 shows the seventh example of the disposition of the sensor of the invention;

FIG. 9 shows an example of the sensor of the invention disposed in a protective garment;

FIG. 10 shows an example of the medical device of the invention disposed in a protective garment;

FIG. 11 shows an example of the invention combined with external detecting devices and automatic disposition;

FIG. 12 shows a flow chart of the invention;

FIG. 13 shows a flow chart of the invention;

FIG. 14 shows a flow chart of the invention;

FIG. 15 shows a flow chart of an example of the invention; and

FIG. 16 shows a flow chart of an example of the invention.

### DETAILED DESCRIPTION OF THE INVENTION

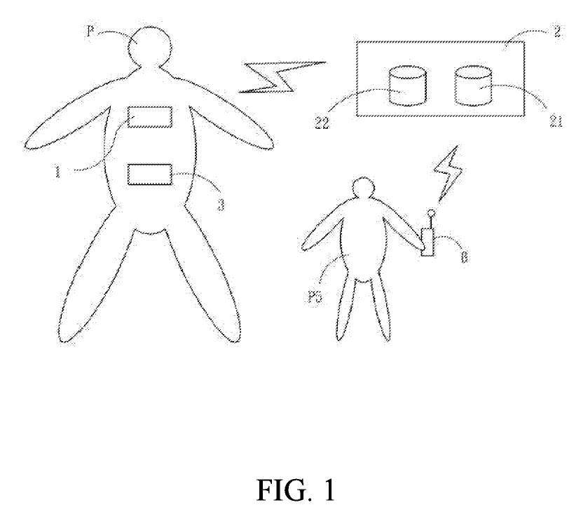

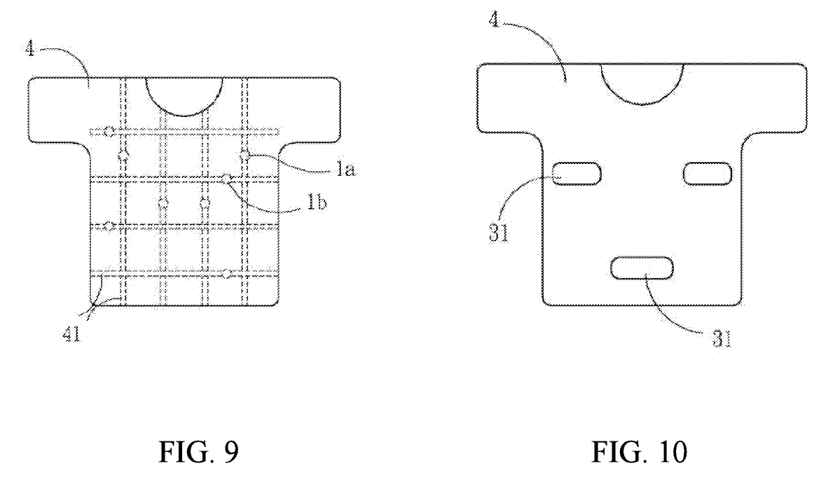

Please refer to FIG. 1, the invention provides an apparatus for monitoring body temperature, respiration, heart sound, swallowing, and medical inquiring. As shown in FIG. 1, the apparatus of the invention includes a sensor 1, a monitor device 2, and a medical device 3. The sensor 1 is disposed on a monitoring location of an user P's body for delivering the information of physical conditions of user P. The monitoring device 2 can receive the information of physical conditions of the user sent by the sensor, and performs further comparison of the received information with pre-stored data to determine the user's condition. Moreover, the monitoring device 2 can warn or guide the user or a medical personnel via image, sound, or text. The medical device 3 can be disposed in a protective garment 4 (as shown in FIG. 9 and FIG. 10), and worn by user P, for performing suitable rescuing measures to user P according to orders sent by the monitoring device 2.

The above-mentioned sensor 1 includes a temperature sensing device 1a, or a respiration/heart sound/swallowing sensing device 1b. The disposing position can be flexibly adjusted based on the physical function desired by the user or medical personnel, to provide suitable information. For example, the sensor can be disposed in the mouth, on the ear or in garments to sense the body temperature or respiration of the user, respectively, whereas it can also be disposed on the ear or the neck to sense the body temperature, respiration, heart sound, and swallowing of the user. Several examples are given as follows.

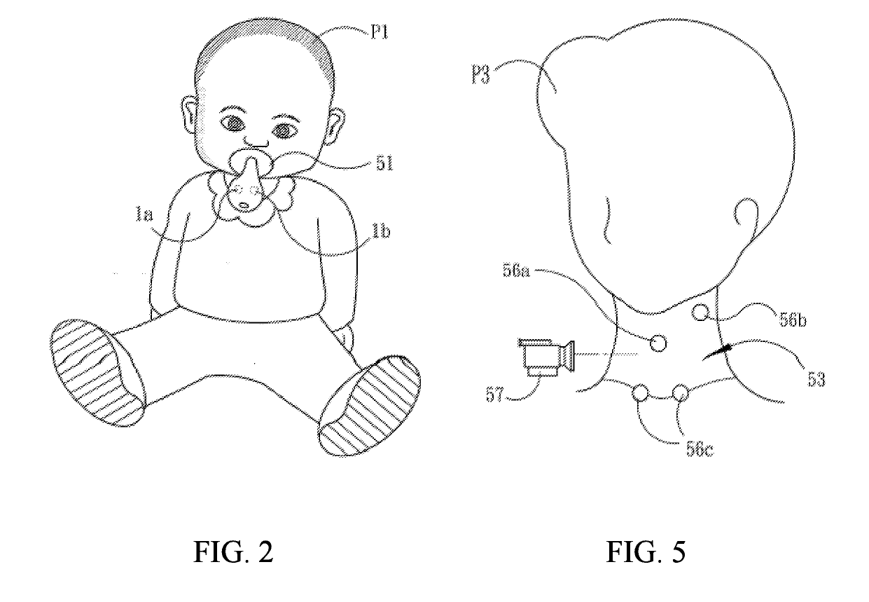

Please refer to FIG. 2, the sensor 1 can be disposed on a pacifier 51, for monitoring the health condition of a child P1. Because the pacifier 51 is put in the child's mouth, it can sense the mouth temperature or respiration of the child P1 directly. Therefore, in the embodiment, the sensor 1 can include temperature sensing device 1a and respiration/heart sound/swallowing sensing device 1b. Moreover, the respiration/heart sound/swallowing sensing device 1b includes a sound amplifier for recording the heart sound, the rate and the strength of respiration, the amplitude of heart sound and respiration, vital capacity, and metabolic rate. The sensor for measuring the metabolic rate is constructed by an oxygen concentration sensor and flow capacity sensor, and the principle of the sensors is to integrate the exhale oxygen concentration and inhale oxygen concentration with the flow capacity, respectively, and then calculate the deviation between them. Moreover, the sensor 1 can further be performed as a conduit, disposed in the user's mouth, for supplying essential substances to the user. The conduit can be disposed with a flow rate counter, for determining the vital capacity, the metabolic rate, and the concentration of CO₂ by monitoring the user's breath.

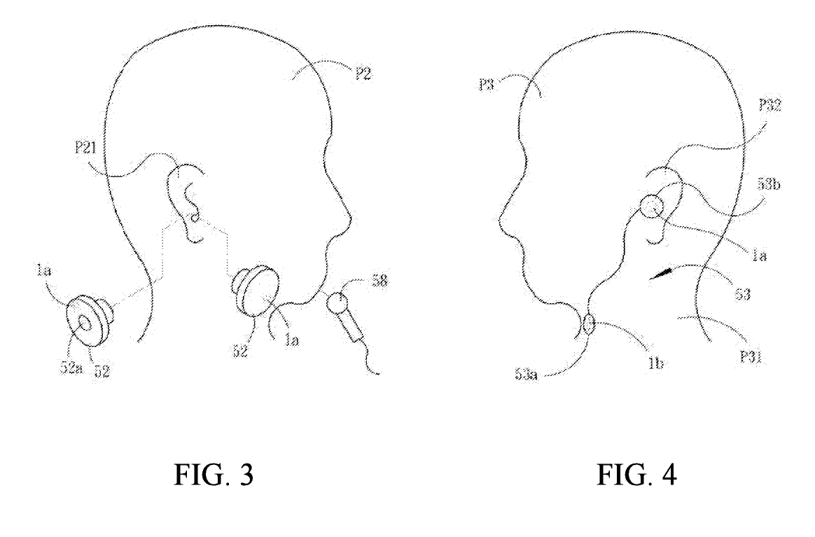

Please refer to FIG. 3, the sensor 1 can be disposed on an earphone 52, and the sensor 1 mainly includes a temperature sensing device 1a, for detecting the temperature of the user (P2) from his ear P21. The sensor can also includes a microphone 58 and a respiration/heart sound/swallowing sensing device 1b, for detecting heart sound, respiration and swallowing of the user, and can pronounce to communicate with the user P2. Moreover, the earphone 52 can be a general earphone or a hollow-type earphone 52a, which can avoid obstructing the user (P2)'s hearing. However, the cost of the hollow-type earphone 52a is higher than the general earphone, the choice of the earphone depends on the requirement of the user P2.

Please refer to FIG. 4, the sensor 1 can be disposed on a conductive microphone 53. The microphone 53a is fitted on the throat portion P31 of an user P3, whereas the receiver 53b is disposed at the ear portion P32 of the user P3. Moreover, the microphone 53a is mainly a heart sound, respiration, and swallowing sensing device 1b, for gathering information of heart sound, respiration, and swallowing from the throat portion P31 of the user P3, and the receiver 53b can mainly be a temperature sensing device 1a.

Please refer to FIG. 5, the sensor 1 can be further disposed on the throat portion of a user. As shown in FIG. 5, the apparatus can further include a throat portion microphone 56a, an accelerometer 56b, and a laryngograph 56c. In the embodiment as shown in FIG. 5, the sensors can measure respiration, heart beat, heart sound, times, rate and cycles of swallowing, and electromyogram (EMG). Moreover, in this embodiment, the sensors can be used to determine if the user P3 is choked, and the apparatus of the invention can inform a medical personnel or a doctor to rescue the user P3 in time. Furthermore, through the throat portion microphone 56a, the user can interact with a doctor in the distal (distant?) end to report his or her situation. A rotating micro CCD camera 57 can be disposed at the throat portion of the user for the doctor to observe the user's neck and chest to assist the determination of the user's situation.

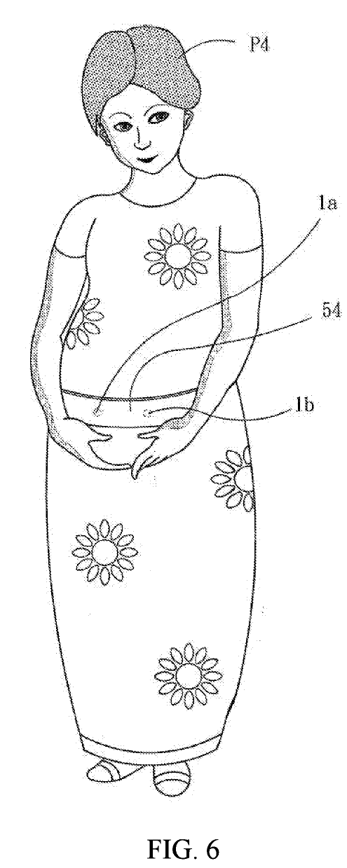

Please refer to FIG. 6, the sensor 1 can be disposed on a belt 54 by clipping or hooking, adhering, inserting to the belt, or disposed in the underwear. The above-mentioned sensor 1 includes a sensing device 1a for temperature and a sensing device 1b for heart sound, respiration and swallowing. Moreover, the sensing device 1b for heart sound, respiration and swallowing is mainly constructed by a sound amplifier, therefore, it can also be used to measure the heartbeat or heart sound of the fetus of an expectant mother P4. Furthermore, because the body temperature of a woman rises during ovulation period or pregnancy, the sensor 1 of the invention can be applied to measure the basal body temperature at a suitable time (such as the sleeping time), and conduct continuous measure for days. If the basal body temperature suddenly rises, the apparatus of the invention can determine the ovulation day is coming and informs the user to be prepared for insemination. On the other hand, if the basal body temperature suddenly rises and the menses period absences, the apparatus may determine a pregnancy situation.

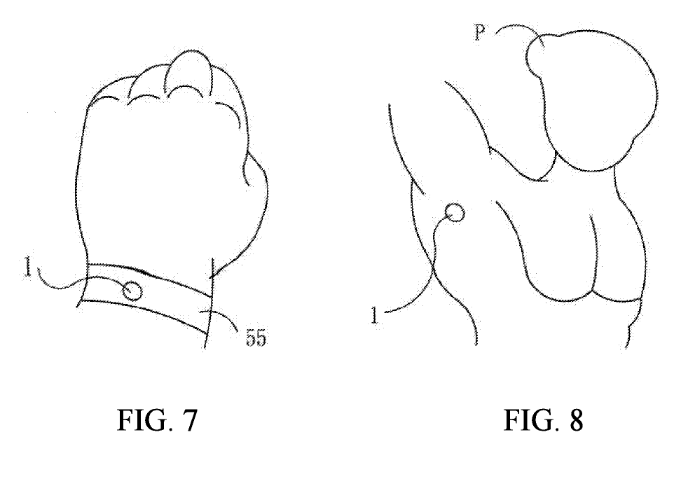

Please refer to FIG. 7, the sensor 1 can be disposed on a wrist watch or wrist sphygmomanometer 55. Moreover, the sphygmomanometer 55 can also perform its original function of measuring the user's blood pressure.

Please refer to FIG. 8, the sensor 1 can be disposed at a user (P)'s armpit, for measuring the respiration, heartbeat, heart sound, body temperature, and swallowing information of the user P.

Please refer to FIG. 9, the sensor 1 can be disposed in a protective garment 4, and can include a temperature sensing device 1a and a heart sound, respiration and swallow sensing device 1b. Particularly, the sensor 1 can be fixed in the protective garment 4 by mechanical means or gas-filled means, and contacts the surface of user's body to measure suitable physical parameters. Because the protective garment 4 covers a larger area of the user's body, the suitable position for disposing the sensors is enlarged, a plurality of guiding tracts 41 can be disposed in the protective garment 4, and the sensor 1a, 1b can be disposed on the guiding tracts 41. Furthermore, the monitoring device 2 can adjust the position of the sensors 1a, 1b on the guiding tracts 41 by sending them control orders. Furthermore, the sensors 1a, 1b can also be disposed inside the protective garment 4 by clipping, hooking, adhering, or inserting in the protective garment 4 as described in the prior arts, and discussion of unnecessary details will be omitted.

Moreover, the above-mentioned sensor 1 can also be an ultrasonic sensor for monitoring lung functions (e.g., times, frequency, and amplitude of respiration) and heart functions. If the sensor 1 is disposed on an expectant mother, it can also be used to measure the fetus' heart functions.

User can apply the apparatus of the invention and dispose one or more sensors 1 as mentioned before on suitable position of the user's body under a doctor's instruction, for gathering physical information to assist the doctor in making more accurate determination.

Please refer to FIG. 1 again, the monitoring device 2 can be a communicating equipment, such as a cell phone, a GPS, a GPRS, a radio, a PDA, a pager, a phone or a computer/ Therefore, the monitoring device 2 can receive each user's data in the same area. Furthermore, the monitoring device 2 has a comparing database 21 and an user database 22. The comparing database 21 stores a plurality of standard physical data to be compared with the information provided by the sensor; and the user database 22 stores a personal medical history of the user P and can be provided to the doctors for further research and application. Moreover, the monitoring device 2 can interact with a distance medical center, for updating the information of the user P.

Please refer to FIG. 10, a medical device 3 is disposed in the protective garment 4. The medical device 3 can be a pressed airbag 31, which can press against the user P after filled with air. Therefore, the pressing airbag 31 can perform Heimlich Maeuver or CPR to the user P. When the user's bronchus is obstructed by sputum, the medical device 3 can adjust the position and rescue the user under the control of the monitoring device 2 by clapping the user's back. Moreover, the medical device 3 can also be an ultrasonic vibrating device or a mechanical vibrating device, and performs the clapping function by ultrasonic vibration or mechanical vibration.

Additionally, the above-mentioned medical device 3 can also be an electric shock providing device, a pain generating device, an oxygen providing device, a steam providing device, a sputum extracting device, a temperature adjusting device, or a drug supplying device. The electric shock providing device can rescue the user by giving an electric shock; the pain generating device can stimulate the user to wake up; the oxygen providing device and the steam providing device can provide the user with oxygen and steam, respectively; the sputum extracting device can remove sputum from user's mouth and throat, and can further collect the sputum for testing or culturing; the temperature adjusting device is disposed inside the protective garment 4, for exchanging thermal energy, and the water in the temperature adjusting device can provide the user with drinking water; and the drug supplying device can supply medication such as bronchodilator, or injection agent, such as steroids, or adhesive treatment, such as temperature-abating paste or pain-relieving paste, which can be disposed under the pressing airbag 31, and paste the skin of the user when the airbag 31 is filled with air, or can be applied by the medial personnel P5 when instructed. Certainly, other suitable medical equipments can be incorporated in the medical device of the invention to provide better service to the user.

Again, please refer to FIG. 1, besides the user P, the apparatus of the invention can further provide instruction to medical personnel P5 chosen from family members, friends, neighbors or professional medical aids. Furthermore, the medical personnel P5 holds a receiver 6, such as mobile phone, GPS, GPRS, radio, infrared rays device, PDA, pager, phone, or a computer. When the monitoring device 2 notices a negative condition on the user P, it can inform the medical personnel P5 in time to rescue the user P. Moreover, the medical personnel P5 can user the receiver 6 to contact with a doctor in the distance, and the doctor can instruct the medical personnel P5 on measures to be taken to rescue the user P via image, sound or text provided by the device.

If the user P or the medical personnel P5 finds a serious condition or the user P has a pre-determined syndrome, the monitoring device 2 can connect with the doctor in the distance to rescue the user P. Moreover, the invention further includes an emergency button, designed to be triggered by a specific criterion to send an emergency message to the monitoring device 2. For example, the specific criterion includes a specific sound of the user P, such as the voice of “help”. The emergency button, however, comes with a cancel button. The user can demand the monitoring device 2 to ignore the emergency message by pressing on the cancel button if he or she presses the emergency button by accident.

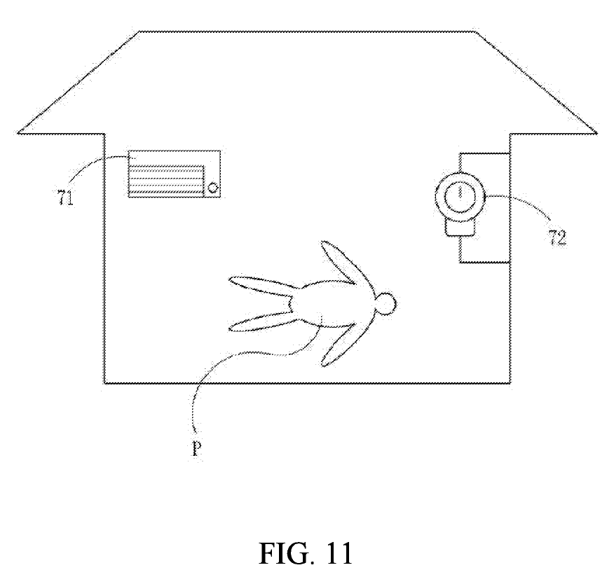

Please refer to FIG. 11, because of the development in technology, many automatic equipment can be found in our houses. Therefore, the monitoring device 2 can perform automatic control by detecting external environment. For example, when the monitoring device 2 detects abnormality in the body temperature of user P, it can control the automatic air conditioner 71 and adjust the environment temperature. Furthermore, if the monitoring device 2 detects the user P absorbs a lot of fuel gas, it can further shot down the fuel gas equipment 72 and electronic equipments to avoid dangerous situations.

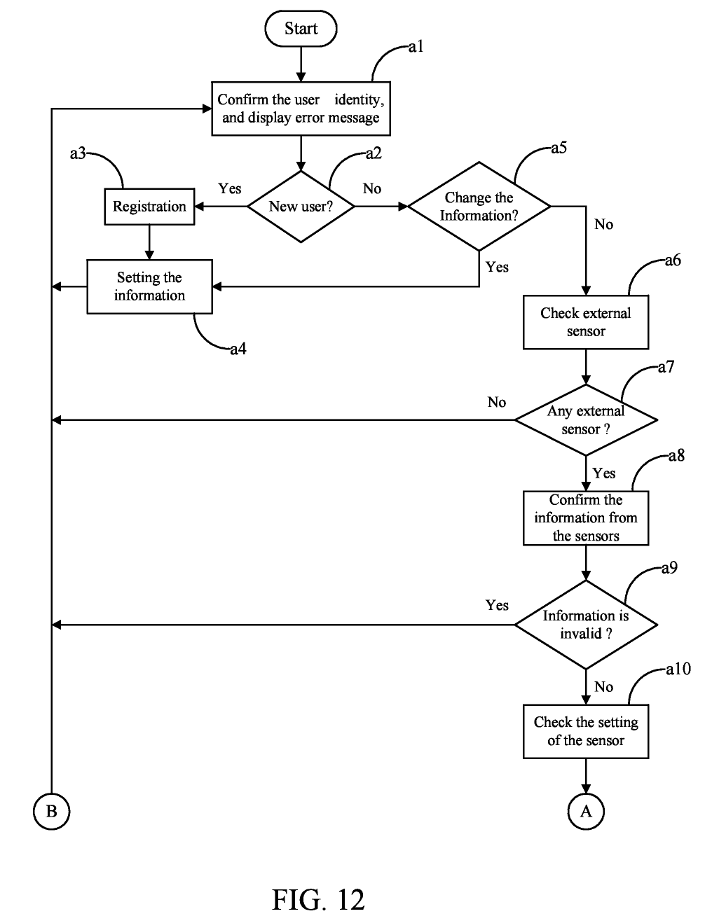

Please refer to FIG. 12, the method for operating the apparatus of the invention is described as follows:

a1. confirm the user's identity, and display error message when the user's identity is incorrect;

a2. determine if the user is a new user;

a3. if the user is a new user, perform a registration program;

a4. during the registration, ask the new user to enter basic information, such as name, age, sex, family, height, weight, address, medical history, family or genetic medical history, person to contact in emergency and his contact information, such as phone number, address, e-mail, ICQ, or MSN;

a5. if the user is not a new user, ask the user if he or she wants to change the information, such as the voice of the user, the figure of apnea, and if the user wants to change the information, process step a4, or process step a6;

a6. connect related detecting devices, such as ultraviolet sensor, temperature sensor, humidity sensor, smoke sensor, CO sensor, and CO₂ sensor, or power switches of air conditioner, doors, windows, lights, video generating equipments, sound generating equipments, text generating equipments, or the warning devices of vehicles, so as to control these devices or equipments via the monitoring device;

a7. check if there is any external sensor, if no, return to the step a1, and if yes, process the step a8;

a8. confirm the information from the sensors;

a9. determine whether the information is invalid, if yes, return to the step a1, and if no, process step a10;

a10. confirm the information by checking the setting of the sensor.

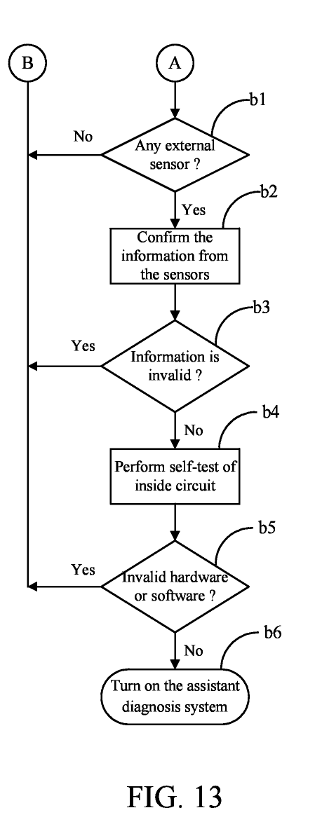

After finishing the steps of FIG. 12, please refer to FIG. 13 for further steps as follow:

b1. check if there is a physical condition sensor, if no, return to step a1, and if yes, process step b2;

b2. confirm the signal of each sensor;

b3. determine if the signal is invalid, if yes, return to step a1, and if no, process step b4;

b4. perform self-test of inside circuit of the apparatus;

5. determine if the hardware and software of the apparatus is invalid, if yes, return to step a1, and if no, process step b6;

b6. turn on the assistant diagnosis system to assist diagnosis and treatment.

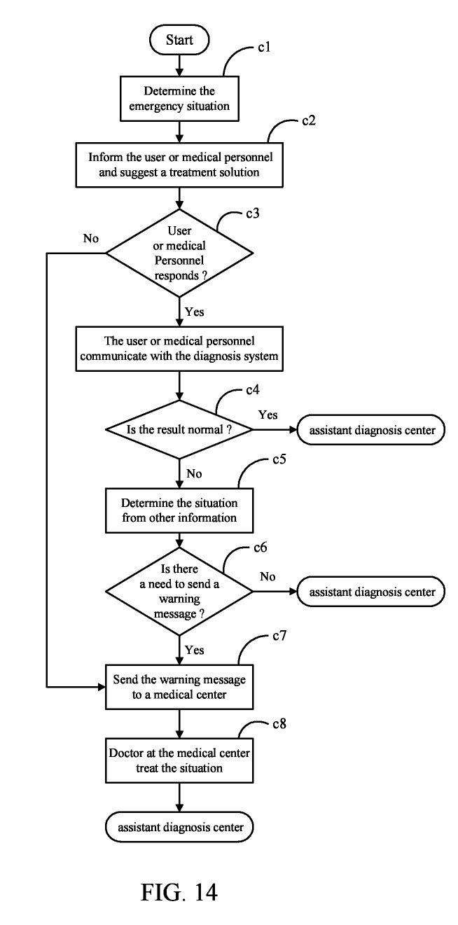

Please further refer to FIG. 14, which shows the steps of emergency rescue. The steps of emergency rescue are described below:

c1. determine the emergency situation, monitor the external abnormal condition (such as leak of fuel gas), and then monitor the physical abnormal condition (such as body temperature detected at 39° C.);

c2. inform the user or medical personnel and suggest a treatment solution, if no one responds, send an emergency message;

c3. determine if the user or medical personnel responds to the emergency message, if yes (means there is someone aware), let the user or medical personnel communicate with the diagnosis system to get suggestions (e.g., ice packing or turn on a cooling device) they need, and if no, connect the emergency medical system to treat the condition;

c4. give feed-back to the result of the treatment, if the result is normal, connect the assistant diagnosis center, and if the result is abnormal, connect the emergency medical system to treat the condition;

c5. determine the situation from other information (evaluate other physical functions);

c6. determine if there is a need to send a warning message;

c7. send the warning message to a medical center;

c8. doctor in the medical center receives the message and further guides the user or medical personnel to treat the condition or to send the user to hospital.

Moreover, the above-mentioned medical treatment should be performed by a doctor to avoid medical disputes, and the personal information can only be modified under the instruction of a doctor or an authorized person.

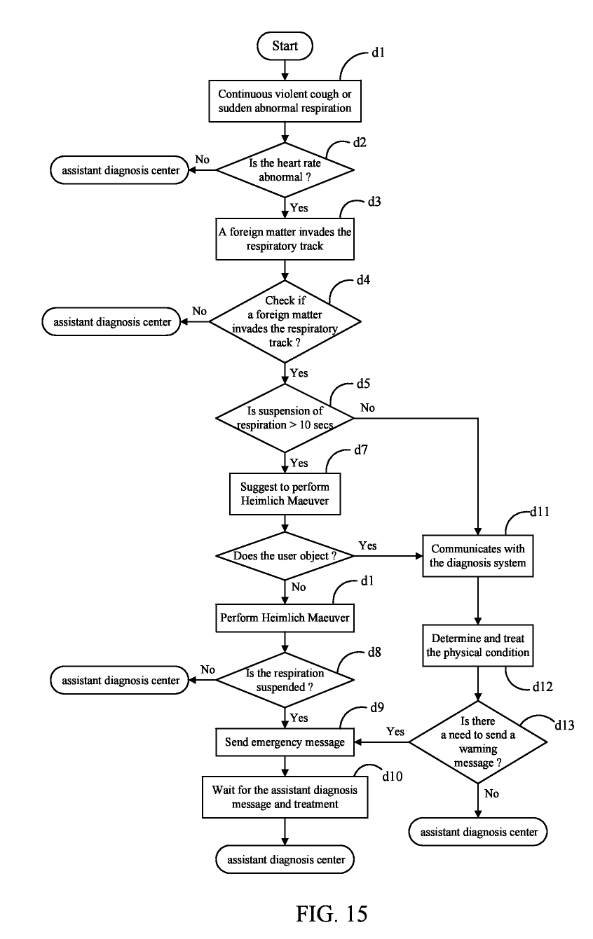

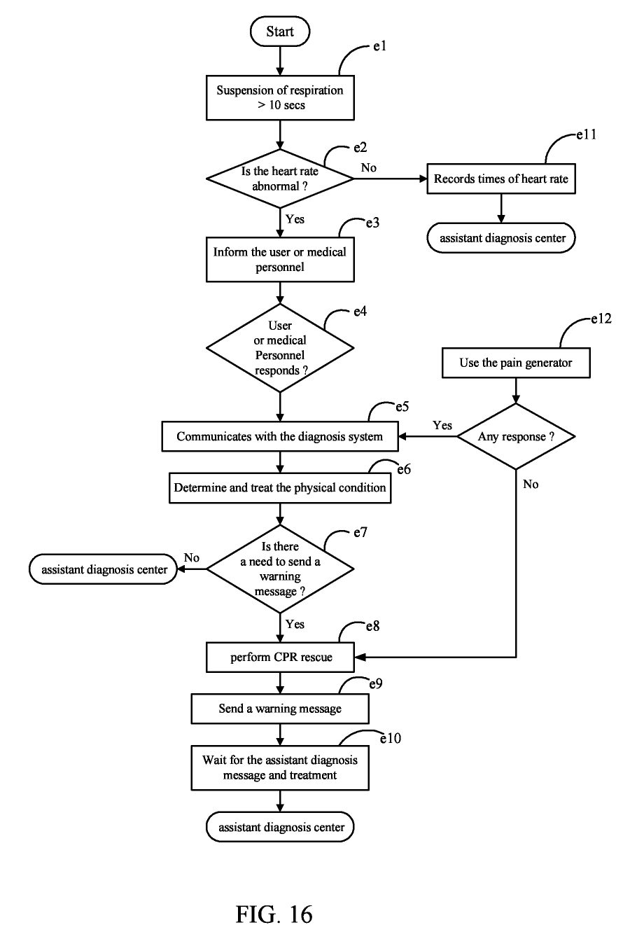

Please refer to FIG. 15 and FIG. 16 for two embodiments of the invention. As show in FIG. 15, the first example is described as follow:

d1. observe continuous violent cough in user or sudden abnormal respiration;

d2. determine if the heart rate of the user is abnormal, if yes, process step d3, and if not, transfer to the assistant diagnosis center;

d3. doubt if a foreign matter invades the respiratory tract;

d4. ask the user to speak, or ask the medical personnel to confirm if a foreign matter invades the respiratory tract, if yes, process step d5, and if not, transfer to the assistant diagnosis center;

d5. determine if the suspension of respiration is longer than 10 secs.;

d6. if the suspension of respiration is longer than 10 secs., suggest the user or the medical personnel to perform Heimlich Maeuver to rescue the user;

d7. perform Heimlich Maeuver to rescue the user;

d8. detect if the user has respiration, if not, transfer to the assistant diagnosis center, and if yes, process step d9;

d9. send emergency message;

d10. wait for the assistant diagnosis message and treatment, and transfer to the assistant diagnosis center.

Additionally, if the suspension of respiration is not longer than 10 secs., or the user or medical personnel objects to perform Heimlich Maeuver, process the following steps:

d11. the user or the medical personnel communicates with the diagnosis system;

d12. determine and treat the physical condition information;

d13. determine if there is a need to send a warning message, if yes, process step d9, and if not, transfer to the assistant diagnosis center.

Another example is described as follows:

e1. sense the respiration, and if the termination of respiration is longer than 10 sec., process step e2;

e2. determine if the heart rate is abnormal, if not, record the heart rate e11, and transfer to the assistant diagnosis center, and if yes, process step e3;

e3. inform the user or medical personnel;

e4. determine the consciousness of the user, if the user is conscious, process step e5, and if the user is unconscious, stimulate the user with pain generator e12, and determine the consciousness of the user again, if the user is still unconscious, process step e8, and if the user is conscious, process step e5;

e5. the user or the medical personnel communicates with the diagnosis system;

e6. determine and treat the physical condition information;

e7. determine if there is a need to send a warning message, if yes, process step

e8, or transfer to the assistant diagnosis center;

e8. perform CPR rescue;

e9. send a warning message;

e10. wait for the assistant diagnosis message and treatment, and transfer to the assistant diagnosis center.

The apparatus for monitoring body temperature, respiration, heart sound, swallowing, and medical inquiring provided by the invention applies a two-way interactive model, so as to monitor the physical conditions of an user without disturbing the user's life, and immediately rescue the user under an emergency condition to reduce the loss of life caused by a waste of time.

With the example and explanations above, the features and spirits of the invention will hopefully be well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. A method for monitoring body temperature, respiration, heart sound, swallowing, medical inquiring, and offering immediate rescue, comprising the steps of: disposing a suitable physical sensor in an user's garment; wearing suitable medical device; detecting the physical conditions of the user, and sending the results monitored to a monitoring device when the physical condition meet a criterion; the monitoring device, pre-stored physical data, comparing the results monitored with the pre-stored data, to determine the physical condition of the user; and displaying a warning or an instruction via image, sound, or text to the user; the monitoring device sending the results monitored to a distal-end medical system, for updating a personal data of the user.

2. The method of claim 1, wherein the monitoring device is pre-connected with a warning device to control the warning device, wherein the warning device comprises ultraviolet sensor, temperature sensor, humidity sensor, smoke sensor, CO sensor, and CO₂ sensor, or power switches of air conditioner, doors, windows, lights, video generating equipments, sound generating equipments, text generating equipments, and the warning devices of vehicles.

3. The method of claim 1, further comprising the step of: choosing a medical personnel from the family members, friends, neighbors, or professional medical aids for helping the user under the instruction provided by the monitoring device.

4. The method of claim 1, wherein when the user is unconscious or under emergency condition, the monitoring device contacting with doctors at an distal-end or the medical system for emergency help, and determining the position of the user by GPS.

5. The method of claim 1, wherein a plurality of users are capable of using a unique outputting device to contact with the distal-end medical system.

6. The method of claim 1, further comprising the step of: measuring and continuously monitoring the date of ovulation or pregnancy, for determining if a woman is pregnancy.

7. The method of claim 1, further comprising the step of: measuring an environment temperature and a body temperature at the same time.

8. The method of claim 1, wherein when the monitoring device detects an abnormal swallowing of the user, the monitoring device noticing the user to stop breathing when the user is swallowing.

9. The method of claim 1, wherein the user and the medical personnel are capable of communicating with the monitoring device.

10. A medical apparatus, for monitoring body temperature, respiration, heart sound, swallowing, medical inquiring, and offering immediate rescue, comprising:
a sensor, disposed on a monitoring position of an user, for delivering an information of physical condition of the user;
a monitoring device, for receiving the information of physical condition of the user, and comparing the information with a pre-stored data to determine the user's condition, and warning or guiding the user or a medical personnel via image, sound, or text; and
a medical device, worn by the user, for performing suitable rescue to the user according to an order sent by the monitoring device.

11. The medical apparatus of claim 10, wherein the sensor is a temperature sensor, disposed in a pacifier, an earphone, a conductive microphone, a protective garment, a belt, an elastic belt of underpants, or an underwear, for measuring a reference temperature of the user.

12. The medical apparatus of claim 10, wherein the sensor is a respiration/heart sound/swallowing sensing device, disposed in a pacifier, an earphone, a conductive microphone, a protective garment, a belt, an elastic belt of underpants, or an underwear, the sensor comprising a sound amplifier, for recording the frequency, strength, time, amplitude of respiration and swallowing and further measuring the heartbeat and heart sound.

13. The medical apparatus of claim 10, wherein the sensor is disposed in a pacifier, for monitoring the mouth temperature, heart sound, respiration and swallowing, and determining the flow rate of breath.

14. The medical apparatus of claim 11, wherein the sensor is disposed in the pacifier, for monitoring the mouth temperature, heart sound, respiration and swallowing, and determining the flow rate of breath.

15. The medical apparatus of claim 10, wherein the sensor is disposed in a belt, for monitoring the body temperature and respiration of the user, wherein when the user is an expectant mother, the sensor is capable of monitoring the heart sound of the fetus.

16. The medical apparatus of claim 11, wherein the sensor is disposed in the belt, for monitoring the body temperature and respiration of the user, wherein when the user is an expectant mother, the sensor is capable of monitoring the heart sound of the fetus.

17. The medical apparatus of claim 10, wherein the sensor is disposed in an earphone, a conductive microphone, and a protective garment.

18. The medical apparatus of claim 11, wherein the sensor is disposed in the earphone, the conductive microphone, and the protective garment.

19. The medical apparatus of claim 10, wherein the sensor is disposed on a guiding tract inside the protective garment, and the monitoring device is capable of adjusting the position of the sensor on the guiding tract.

20. The medical apparatus of claim 10, wherein the sensor is disposed in a protective garment, an underpants, and an underwear by clipping or hooking, adhering, inserting to the protective garment, the underpants, and the underwear, and the sensor is capable of being adjusted its position.

21. The medical apparatus of claim 10, wherein the monitoring device further comprises a comparing database and an user database, wherein the comparing database stores a plurality of standard physical data to be compared with the information provided by the sensor; and the user database storing a personal medical history of the user.

22. The medical apparatus of claim 10, wherein the medical device is a pressed airbag, for performing Heimlich Maeuver of CPR to the user, or clapping the user's back when the user's bronchus is obstructed by sputum.

23. The medical apparatus of claim 10, wherein the medical device is an electric shock providing device, for rescuing the user by giving an electric shock.

24. The medical apparatus of claim 10, wherein the medical device is a pain generating device, for stimulating the user to wake up.

25. The medical apparatus of claim 10, wherein the medical device is an oxygen providing device, for providing the user with oxygen.

26. The medical apparatus of claim 10, wherein the medical device is a sputum extracting device, for removing sputum from the user's mouth and throat, and further collecting the sputum for testing or culturing.

27. The medical apparatus of claim 10, wherein the medical device is a steam providing device, for treating the user with steam.

28. The medical apparatus of claim 10, wherein the medical device is a drug supplying device, for providing oral medication, injection agent, or adhesive treatment.

29. The medical apparatus of claim 10, wherein the sensor comprises a conduit, disposed in the user's mouth, for supplying essential substances to the user, and the conduit is capable of being disposed with a flow rate counter, for determining the vital capacity, the metabolic rate, and the concentration of CO₂ by monitoring the user's breath.

30. The medical apparatus of claim 10, further comprising a transceiving device, carried by the user and medical personnel, for receiving the image, sound, and text sent by the monitoring device, and sending the results detected by the sensor to the monitoring device, and the transceiving device is a mobile phone, a GPS device, a GPRS device, a radio, an infrared rays device, a PDA, a pager, a phone, a calculator, or a Bluetooth device.

31. The medical apparatus of claim 10, further comprising an emergency switch, for sending an emergency message to the monitoring device when triggered by a specific criterion, which comprises a specific voice of the user.

32. The medical apparatus of claim 10, wherein the medical device is a temperature adjusting device, disposed in a protective garment, for exchanging thermal energy, and the water in the temperature adjusting device is capable of providing the user with drinking water.

33. The medical apparatus of claim 10, wherein the sensor is fixed in the protective garment by mechanical means or gas-filled means, so as to contact the skin surface of the user.

34. The medical apparatus of claim 10, further comprising a rotatable video camera, disposed at the throat portion of the user, for a doctor to observe the user's neck and chest to assist the determination of the user's situation.
